(19) United States
(12) Patent Application Publication
(10) Pub. No.: US 2009/0023698 A1
(43) Pub. Date: Jan. 22, 2009

(54) METHODS OF MANUFACTURING BIOACTIVE 3-ESTERS OF BETULINIC ALDEHYDE AND BETULINIC ACID

(76) Inventors: Pavel A. Krasutsky, Duluth, MN (US); Oksana Kolomitsyna, Duluth, MN (US)

Correspondence Address: SCHWEGMAN, LUNDBERG & WOESSNER, P.A., P.O. BOX 2938, MINNEAPOLIS, MN, 55402, US

(21) Appl. No.: 11/910,149

(22) PCT Filed: Mar. 29, 2006
(86) PCT No.: PCT/US06/11793

§ 371 Date: Oct. 1, 2008

Related U.S. Application Data
(60) Provisional application No. 60/666,024, filed on Mar. 29, 2005.

(51) Int. Cl. C07J 63/00; A61K 31/56

(52) U.S. Cl. 514/182; 552/511

(57) ABSTRACT

The present invention provides a method for preparing a compound of formula (I), the method comprising contacting a compound of formula (II) with an effective amount of a compound of formula (III) or (IV). The present invention also provides a method for preparing a compound of formula (VI), the method comprising contacting a compound of formula (II) with an effective amount of one or more of 2,2-dimethylsuccinic acid, 2,2 dimethylbutanedioyl dichloride, 2,2-dimethylbutanedioyl dibromide, and 2,2 dimethylsuccinic anhydride. The present invention also provides a compound obtained from the method of the present invention.

### BACKGROUND OF THE INVENTION

New sources of therapeutic and cosmetic agents are needed to reduce heath care costs in the United States and in society generally. Plant-derived natural products are a proven source of effective therapeutic and cosmetic agents. Widely recognized examples of natural product drugs include paclitaxel (Taxol®) and camptothecin. Useful natural product derivatives can be produced by chemically modifying naturally occurring compounds. More efficacious derivatives can be produced by such modifications of the structure of the naturally occurring compound.

Betulin is a pentacyclic triterpenoid isolated from the outer bark of paper birch trees (Betula paperifera). Betulin can be found in the bark of the white birch in concentrations of up to about 24 wt. %. United States pulp mills that process birch trees produce enough bark waste to allow for the inexpensive isolation of ton-scale quantities of these triterpenoids. As such, betulin could serve as an advantageous source of therapeutic and cosmetic compound derivatives.

Several triterpenes and triterpene derivatives, including betulin derivatives, have known medical applications. Various triterpenes with antibacterial activity were disclosed by Krasutsky et al. (U.S. Pat. No. 6,689,767). Betulin and related compounds with anti-viral activity against herpes simplex virus were disclosed by Carlson et al. (U.S. Pat. No. 5,750,578). Studies have also shown that betulinic acid and betulinic acid derivatives can inhibit various types of cancer cells, such as neuroblastoma and melanoma. Das Gupta et al. (U.S. Pat. No. 5,658,947), Pezzuto et al. (U.S. Pat. No. 5,962,527) and Anderson et al. (WO 95/04526). Some of these triterpenoids have been found to inhibit the enzymatic synthesis of polyamines, which are required for optimum cell growth, thereby inhibiting the growth of the targeted cells.

Current methods of modifying natural products have drawbacks, including the use of toxic reagents, low conversions (i.e., yields), and methods that are often not amenable to large-scale industrial synthesis. See, e.g., U.S. Pat. No. 5,679,828. Ideally, new therapeutic and cosmetic agents would be derived from an abundant source and would be inexpensive to manufacture.

New agents that are active against bacteria, fungi, viruses, and cancer are needed. Also needed is a source of agents that can be conveniently and inexpensively converted to therapeutic and cosmetic agents. New agents would be less expensive to manufacture if they were derived from abundant natural products. Accordingly, new methods for the synthesis of therapeutic and cosmetic compounds and their precursors from readily available naturally isolated compounds are needed. Additionally, highly efficient methods that can be adapted to large-scale preparation are desired. The present application is directed to meeting these needs by providing useful syntheses of various betulin derivatives.

### SUMMARY OF THE INVENTION

The present invention provides methods of manufacturing bioactive 3-esters of betulinic aldehyde and betulinic acid. The methods are relatively inexpensive, provide relatively high yields, can be carried out on a commercial scale (e.g., kilogram), employ relatively environmentally friendly reagents, and/or employ as starting materials, naturally occurring compounds that are abundant in nature.

The present invention provides a method for preparing a compound of formula (I):

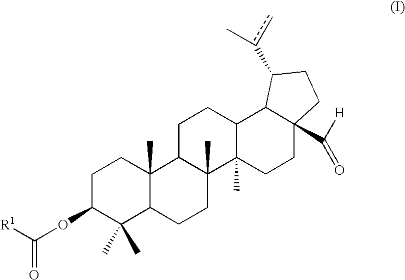

the method comprising contacting a compound of formula (II):

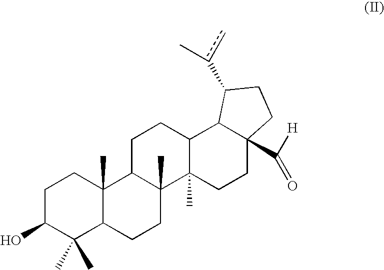

with an effective amount of a compound of formula (III) or (IV):

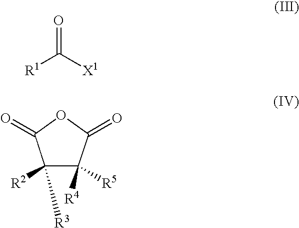

wherein,

R¹ is X¹C(═O)R^{x}—;

R^{x} is alkylene, cycloalkylene, carbocyclene, arylene, heterocyclene, or heteroarylene;

X¹ is hydroxyl, halo, alkoxy or —OC(═O)R^{y};

R^{y} is alkyl, cycloalkyl, carbocycle, aryl, heterocycle, or heteroaryl; and

each of R²-R⁵ is independently H, alkyl, cycloalkyl, carbocycle, aryl, heterocycle, or heteroaryl; and

the bond represented by — is optionally present.

The present invention also provides a method for preparing a compound of formula (VI):

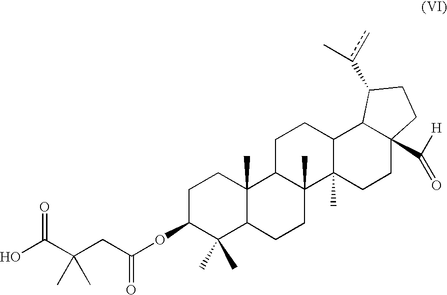

the method comprising contacting a compound of formula (II):

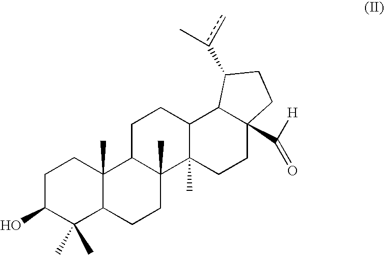

with an effective amount of a compound selected from the group of 2,2-dimethylsuccinic acid, 2,2-dimethylbutanedioyl dichloride, 2,2-dimethylbutanedioyl dibromide, and 2,2-dimethylsuccinic anhydride;

wherein the bond represented by — is optionally present.

The present invention also provides a compound obtained from the method of the present invention.

The present invention provides a pharmaceutical composition that includes a pharmaceutically acceptable carrier and a compound of the present invention.

The present invention also provides a cosmetic composition that includes a cosmetically acceptable carrier and a compound of the present invention.

### DETAILED DESCRIPTION OF THE INVENTION

As used herein, the following terms and expressions have the indicated meanings. It will be appreciated that the compounds of the present invention can contain asymmetrically substituted carbon atoms, and can be isolated in optically active or racemic forms. It is well known in the art how to prepare optically active forms, such as by resolution of racemic forms or by synthesis, from optically active starting materials. All chiral, diastereomeric, racemic forms and all geometric isomeric forms of a structure are intended, unless the specific stereochemistry or isomeric form is specifically indicated.

As used herein, “pharmaceutically acceptable salt” or “physiologically acceptable salt” refer to derivatives of the disclosed compounds wherein the parent compound is modified by making acid or base salts thereof. Examples of physiologically acceptable salts include, but are not limited to, mineral or organic acid salts of basic residues such as amines; alkali or organic salts of acidic residues such as carboxylic acids; and the like. The physiologically acceptable salts include the conventional non-toxic salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The physiologically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Merck Publishing Company, Easton, Pa., 1985, p. 1418, the disclosure of which is hereby incorporated by reference.

The phrase “physiologically acceptable” or “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by and employed in the present invention.

“Substituted” is intended to indicate that one or more (e.g., 1, 2, 3, 4, or 5; preferably 1, 2, or 3; and more preferably 1 or 2) hydrogen atoms on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Alternatively, the suitable indicated groups can include, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR, —S(═O)₂O⁻, —S(═O)₂OH, —S(═O)₂R, —OS(═O)₂OR, —S(═O)₂NR, —S(═O)R, —OP(═O)(OR)₂, —P(═O)(OR)₂, —P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen: F, Cl, Br, or I; and each R is independently H, alkyl, aryl, heterocycle, protecting group or prodrug moiety. When a substituent is a keto (i.e., ═O) or thioxo (i.e., ═S) group, then 2 hydrogens on the atom are replaced.

One diastereomer may display superior activity compared with the other. When required, separation of the racemic material can be achieved by high pressure liquid chromatography (HPLC) using a chiral column or by a resolution using a resolving agent such as camphonic chloride as in Thomas J. Tucker, et al., J. Med. Chem. 1994 37, 2437-2444. A chiral compound may also be directly synthesized using a chiral catalyst or a chiral ligand, e.g. Mark A. Huffman, et al., J. Org. Chem. 1995, 60, 1590-1594.

The term “alkyl” refers to a monoradical branched or unbranched saturated hydrocarbon chain preferably having from 1 to 10 carbon atoms, preferably 1 to 6 carbon atoms, and more preferably from 1 to 4 carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃. The alkyl can be unsubstituted or substituted.

The term “alkenyl” refers to a monoradical branched or unbranched partially unsaturated hydrocarbon chain (i.e. a carbon-carbon, sp² double bond) preferably having from 2 to 10 carbon atoms, preferably 2 to 6 carbon atoms, and more preferably from 2 to 4 carbon atoms. Examples include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂). The alkenyl can be unsubstituted or substituted.

The term “alkynyl” refers to a monoradical branched or unbranched hydrocarbon chain, having a point of complete unsaturation (i.e. a carbon-carbon, sp triple bond), preferably having from 2 to 10 carbon atoms, preferably 2 to 6 carbon atoms, and more preferably from 2 to 4 carbon atoms. This term is exemplified by groups such as ethynyl, 1-propynyl, 2-propynyl, 1-butynyl, 2-butynyl, 3-butynyl, 1-hexynyl, 2-hexynyl, 3-hexynyl, and the like. The alkynyl can be unsubstituted or substituted.

“Alkylene” refers to a saturated, branched or straight chain hydrocarbon radical of 1-18 carbon atoms, and having two monovalent radical centers derived by the removal of two hydrogen atoms from the same or two different carbon atoms of a parent alkane. Typical alkylene radicals include, but are not limited to, methylene (—CH₂—) 1,2-ethyl (—CH₂CH₂—), 1,3-propyl (—CH₂CH₂CH₂—), 1,4-butyl (—CH₂CH₂CH₂CH₂—), and the like. The alkynyl can be unsubstituted or substituted.

“Alkenylene” refers to an unsaturated, branched or straight chain hydrocarbon radical of 2-18 carbon atoms, and having two monovalent radical centers derived by the removal of two hydrogen atoms from the same or two different carbon atoms of a parent alkene. Typical alkenylene radicals include, but are not limited to, 1,2-ethylene (—CH═CH—). The alkenylene can be unsubstituted or substituted.

“Alkynylene” refers to an unsaturated, branched or straight chain hydrocarbon radical of 2-18 carbon atoms, and having two monovalent radical centers derived by the removal of two hydrogen atoms from the same or two different carbon atoms of a parent alkyne. Typical alkynylene radicals include, but are not limited to, acetylene (—C≡C—), propargyl (—CH₂C≡C—), and 4-pentynyl (—CH₂CH₂CH₂C≡CH—). The alkynylene can be unsubstituted or substituted.

The term “alkoxy” refers to the groups alkyl-O-, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like. The alkoxy can be unsubstituted or substituted.

The term “aryl” refers to an unsaturated aromatic carbocyclic group of from 6 to 12 carbon atoms having a single ring (e.g., phenyl) or multiple condensed (fused) rings, wherein at least one ring is aromatic (e.g., naphthyl, dihydrophenanthrenyl, fluorenyl, or anthryl). The aryl can be unsubstituted or substituted.

The term “cycloalkyl” refers to cyclic alkyl groups of from 3 to 10 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like. The cycloalkyl can be unsubstituted or substituted.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, selected from alkyl, alkenyl, alkynyl, alkoxy, halo, haloalkyl, hydroxy, hydroxyalkyl, aryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl and cyano. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

“Heterocycle” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention “heterocycle” includes a “carbocycle” as defined herein, wherein one or more (e.g. 1, 2, 3, or 4) carbon atoms have been replaced with a heteroatom (e.g. O, N, or S).

Examples of heterocycles include, by way of example and not limitation: pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl

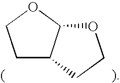

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetraliydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” refers to a saturated, unsaturated or aromatic ring having 3 to 7 carbon atoms as a monocycle, 7 to 12 carbon atoms as a bicycle, and up to about 30 carbon atoms as a polycycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl, adamantly, and naphthyl.

The terms “cycloalkylene”, “carbocyclene”, “arylene”, “heterocyclene”, and “heteroarylene” refer to diradicals of the parent group. For example, “arylene” refers to an aryl diradical, e.g., an aryl group that is bonded to two other groups or moieties.

The term “alkanoyl” refers to C(═O)R, wherein R is an alkyl group as previously defined.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)NH—, wherein R is alkyl or aryl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” refers to —OH.

As used herein, “NaClO₂” refers to sodium chlorite.

As used herein, “KClO₂” refers to potassium chlorite.

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

As used herein, “contacting” refers to the act of touching, making contact, or of bringing to immediate or close proximity, including at the molecular level.

As used herein, “triterpene” or “triterpenoid” refers to a plant secondary metabolite that includes a hydrocarbon, or its oxygenated analog, that is derived from squalene by a sequence of straightforward cyclizations, functionalizations, and sometimes rearrangement. Triterpenes or analogues thereof can be prepared by methods known in the art, i.e., using conventional synthetic techniques or by isolation from plants. Suitable exemplary triterpenes and the biological synthesis of the same are disclosed, e.g., in R. B. Herbert, The Biosynthesis of Secondary Plant Metabolites, 2nd. ed. (London: Chapman 1989). The term “triterpene” refers to one of a class of compounds having approximately 30 carbon atoms and synthesized from six isoprene units in plants and other organisms. Triterpenes consist of carbon, hydrogen, and optionally oxygen. Most triterpenes are secondary metabolites in plants. Most, but not all, triterpenes are pentacyclic. Examples of triterpenes include betulin, allobetulin, lupeol, friedelin, and all sterols, including lanosterol, stigmasterol, cholesterol, β-sitosterol, and ergosterol. Additional examples of triterpenes include those described, e.g., in Published U.S. patent application Ser. Nos. 2004/0097436, 2002/0128210, and 2002/0119935.

As used herein, “betulin” refers to 3β,28-dihydroxy-lup-20(29)-ene. Betulin is a pentacyclic triterpenoid derived from the outer bark of paper birch trees (Betula papyrifera, B. pendula, B. verucosa, etc.). The CAS Registry No. is 473-98-3. It can be present at concentrations of up to about 24% of the bark of white birch. Merck Index, twelfth edition, page 1236 (1996). Structurally, betulin is shown below:

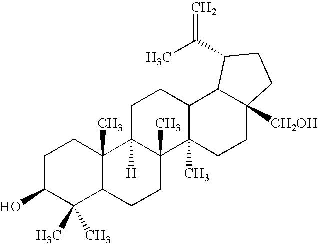

As used herein, “betulinic acid” refers to 3(β)-hydroxy-20(29)-lupaene-28-oic acid; 9-hydroxy-1-isopropenyl-5a,5b,8,8,11a-pentamethyl-eicosahydro-cyclopenta[a]chrysene-3a-carboxylic acid. The CAS Registry No. is 472-15-1. Structurally, betulinic acid is shown below:

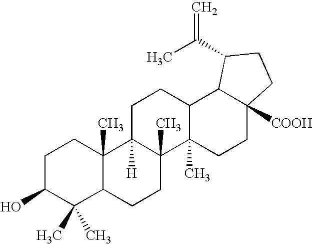

As used herein, “betulin aldehyde” refers to 3(β)-hydroxy-lup-20(29)-en-28-al; 3aH-cyclopenta[a]chrysene, lup-20(29)-en-28-al derivative; betulinaldehyde; betulinic aldehyde; or betunal. The CAS Registry Number is 13159-28-9. Structurally, betulin aldehyde is shown below:

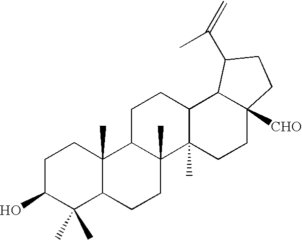

As used herein, “betulin-3-(3′,3′-dimethylsuccinate)-28-al” refers to a compound of the formula:

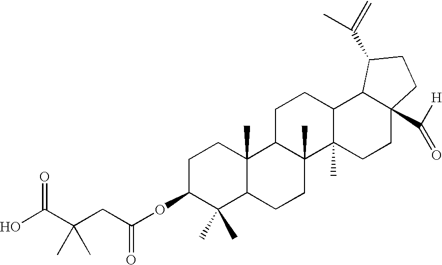

As used herein, “betulin-3-(3′,3′-dimethylsuccinate)-28-carboxylic acid” refers to a compound of the formula:

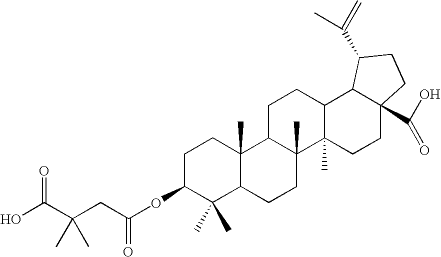

As used herein, “treat” or “treating” refers to: (i) preventing a pathologic condition from occurring (e.g. prophylaxis) or symptoms related to the same; (ii) inhibiting the pathologic condition or arresting its development or symptoms related to the same; or (iii) relieving the pathologic condition or symptoms related to the same.

Utility: The compounds disclosed herein (i.e., those useful in the present invention) can possess suitable biological activity against HIV, herpes, hepatitis, cancer, viral infections, fungal infections, and/or bacterial infections. As such, they are useful as agents for the treatment of HIV, herpes, hepatitis, cancer, viral infections, fungal infections, and/or bacterial infections; and related diseases and symptoms.

The invention can be exemplified by the following enumerated embodiments.

### EMBODIMENT 1

A method for preparing a compound of formula (I)

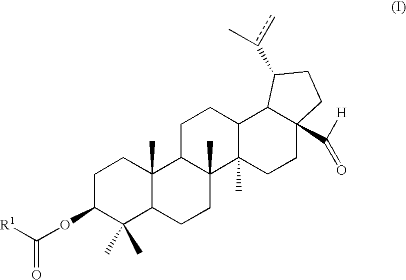

the method comprising contacting a compound of formula (II):

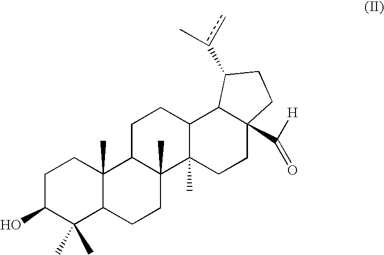

with an effective amount of a compound of formula (III) or (IV):

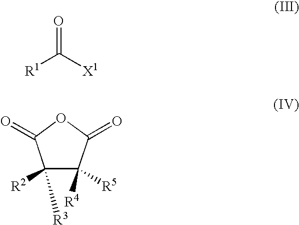

wherein,

R¹ is X¹C(═O)R^{x}—;

R^{x} is alkylene, cycloalkylene, carbocyclene, arylene, heterocyclene, or heteroarylene;

X¹ is hydroxyl, halo, alkoxy or —OC(═O)R^{y};

R^{y} is alkyl, cycloalkyl, carbocycle, aryl, heterocycle, or heteroaryl;

each of R²-R⁵ is independently H, alkyl, cycloalkyl, carbocycle, aryl, heterocycle, or heteroaryl; and

the bond represented by — is optionally present.

### EMBODIMENT 2

The method of embodiment 1, wherein R¹ is HOOCC(CH₃)₂CH₂—.

### EMBODIMENT 3

The method of embodiment 1, wherein R¹ is BrOCC(CH₃)₂CH₂—.

### EMBODIMENT 4

The method of embodiment 1, wherein R¹ is ClOCC(CH₃)₂CH₂—.

### EMBODIMENT 5

The method of any one of embodiments 1-4, wherein R^{x} is —C(CH₃)₂CH₂—.

### EMBODIMENT 6

The method of any one of embodiments 1-5, wherein each X¹is hydroxyl.

### EMBODIMENT 7

The method of any one of embodiments 1-5, wherein each X¹ is bromo.

### EMBODIMENT 8

The method of any one of embodiments 1-5, wherein each X¹ is chloro.

### EMBODIMENT 9

The method of any one of embodiments 1-5, wherein each X¹ is —OC(═O)R^{y}.

### EMBODIMENT 10

The method of embodiment 1, wherein R² is methyl.

### EMBODIMENT 11

The method of embodiment 1, wherein R³ is methyl.

### EMBODIMENT 12

The method of embodiment 1, wherein R⁴ is methyl.

### EMBODIMENT 13

The method of embodiment 1, wherein R⁵ is methyl.

### EMBODIMENT 14

The method of embodiment 1, wherein R² is hydrogen.

### EMBODIMENT 15

The method of embodiment 1, wherein R³ is hydrogen.

### EMBODIMENT 16

The method of embodiment 1, wherein R⁴ is hydrogen.

### EMBODIMENT 17

The method of embodiment 1, wherein R⁵ is hydrogen.

### EMBODIMENT 18

The method of embodiment 1, wherein R² and R³ are each methyl and R⁴ and R⁵ are each hydrogen.

### EMBODIMENT 19

The method embodiment 1, wherein R² and R³ are each hydrogen and R⁴ and R⁵are each methyl.

### EMBODIMENT 20

The method of any one of embodiments 1-19, wherein the contacting is carried out at a temperature of about 10° C. to about 120° C.

### EMBODIMENT 21

The method of any one of embodiments 1-20, wherein the contacting is carried out in a solvent system selected from the group of ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

### EMBODIMENT 22

The method of any one of embodiments 1-20, wherein the contacting is carried out for a period of time of about 30 minutes to about 48 hours.

### EMBODIMENT 23

The method of any one of embodiments 1-20, wherein at least about 10 kg of the compound of formula (I) is obtained.

### EMBODIMENT 24

The method of any one of embodiments 1-20, wherein at least about 85 mol % of the compound of formula (I) is obtained, based upon the compound of formula (II).

### EMBODIMENT 25

The method of any one of embodiments 1-20, wherein the compound of formula (I) is obtained having a purity of at least about 95 wt. %.

### EMBODIMENT 26

The method of any one of embodiments 1-25, further comprising contacting the compound of formula (I) with an effective amount of an alkali metal chlorite, to provide a compound of formula (V):

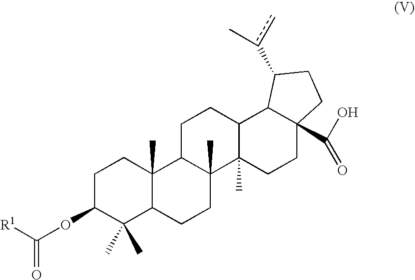

or a pharmaceutically acceptable salt thereof.

### EMBODIMENT 27

The method of embodiment 26 wherein the alkali metal chlorite is NaClO₂, KClO₂, or a combination thereof.

### EMBODIMENT 28

The method of embodiment 26, wherein the contacting is carried out at a temperature of about 10° C. to about 120° C.

### EMBODIMENT 29

The method of embodiment 26, wherein the contacting is carried out in a solvent system selected from the group of ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

### EMBODIMENT 30

The method of embodiment 26, wherein the contacting is carried out for a period of time of about 30 minutes to about 48 hours.

### EMBODIMENT 31

The method of embodiment 26, wherein at least about 10 kg of the compound of formula (V) is obtained.

### EMBODIMENT 32

The method of embodiment 26, wherein at least about 85 mol % of the compound of formula (V) is obtained, based upon the compound of formula (I).

### EMBODIMENT 33

The method of embodiment 26, wherein the compound of formula (V) is obtained having a purity of at least about 95 wt. %.

### EMBODIMENT 34

The method of any one of embodiments 1-33, wherein the bond represented by — is present.

### EMBODIMENT 35

The method of any one of embodiments 1-33, wherein the bond represented by — is absent.

### EMBODIMENT 36

A method for preparing a compound of formula (VI):

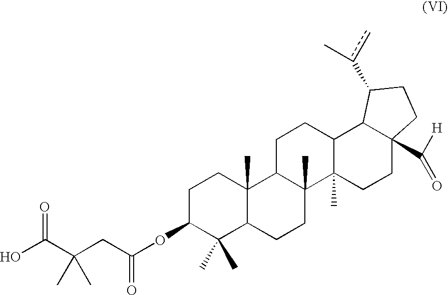

the method comprising contacting a compound of formula (II):

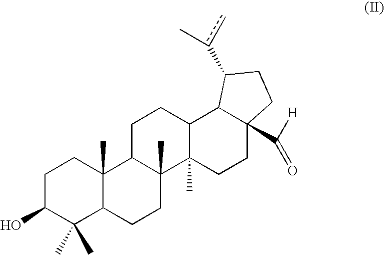

with an effective amount of a compound selected from the group of 2,2-dimethylsuccinic acid, 2,2-dimethylbutanedioyl dichloride, 2,2-dimethylbutanedioyl dibromide, and 2,2-dimethylsuccinic anhydride;

wherein the bond represented by — is optionally present.

### EMBODIMENT 37

The method of embodiment 36, wherein the contacting is carried out at a temperature of about 10° C. to about 120° C.

### EMBODIMENT 38

The method of embodiment 36, wherein the contacting is carried out in a solvent system selected from the group of ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

### EMBODIMENT 39

The method of embodiment 36, wherein the contacting is carried out for a period of time of about of about 30 minutes to about 48 hours.

### EMBODIMENT 40

The method of embodiment 36, wherein at least about 10 kg of the compound of formula (VI) is obtained.

### EMBODIMENT 41

The method of embodiment 36, wherein at least about 85 mol % of the compound of formula (VI) is obtained, based upon the compound of formula (II).

### EMBODIMENT 42

The method of embodiment 36, wherein the compound of formula (VI) is obtained having a purity of at least about 95 wt. %

### EMBODIMENT 43

The method of embodiment 36, further comprising contacting the compound of formula (VI) with an effective amount of NaClO₂ or KClO₂, to provide a compound of formula (VII):

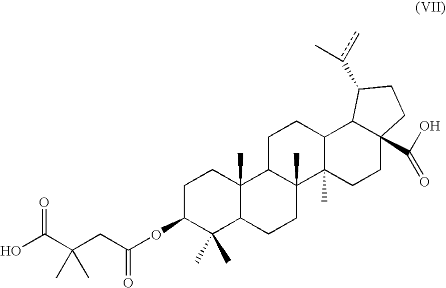

or a pharmaceutically acceptable salt thereof.

### EMBODIMENT 44

The method of embodiment 43, wherein the contacting is carried out at a temperature of about 10° C. to about 120° C.

### EMBODIMENT 45

The method of embodiment 43, wherein the contacting is carried out in a solvent system selected from the group of water, an alcohol, unsaturated hydrocarbons, ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

### EMBODIMENT 46

The method of embodiment 43, further comprising a free halogen scavenger.

### EMBODIMENT 47

The method of embodiment 43, further comprising a halogen scavenger that is an unsaturated hydrocarbon.

### EMBODIMENT 48

The method of embodiment 43, further comprising a halogen scavenger selected from the group of amylene, cyclohexene, methylcyclohexene and cyclopentene.

### EMBODIMENT 49

The method of embodiment 43, wherein the contacting is carried out for a period of time of about 30 minutes to about 48 hours.

### EMBODIMENT 50

The method of embodiment 43, wherein at least about 10 kg of the compound of formula (VII) is obtained.

### EMBODIMENT 51

The method of embodiment 43, wherein at least about 85 mol % of the compound of formula (VII) is obtained, based upon the compound of formula (VI).

### EMBODIMENT 52

The method of embodiment 43, wherein the compound of formula (VII) is obtained having a purity of at least about 95 wt. %.

### EMBODIMENT 53

The method of embodiment 43, wherein the compound of formula (VI) is contacted with an effective amount of NaClO₂ or KClO₂, in the presence of a basic catalyst selected from the group of amines, alkylamines, dialkylamines, trialkylamines, pyridine, NN-dimethylaminopyridine, triethylamine, 2,4,6-collidine, 2,6-lutidine, morpholine, imidazole, PPY(4-pyrrolidinopyridine), and DABCO (1,4-diazabicyclo(2,2,2)octane).

### EMBODIMENT 54

The method of embodiment 43, wherein the compound of formula (VI) is contacted with an effective amount of NaClO₂ or KClO₂, in the presence of a condensation catalyst selected from the group of DCC (N,N-dicyclohexylcarbodiimide), 2,4,6-trichlorobenzoyl chloride, di-2-pyridyl carbonate, diethyl azodicarboxylate and triethylphosphite, 1,2-benzisoxazol-3-yl-diphenylphosphate, N,N-carbonyldiimidazole and 1,8-diazabicyclo[5,4,0]-undec-7-ene, isoureas, benzoxazoles, and benzisothiazoles.

### EMBODIMENT 55

The method of any one of embodiments 36-54, wherein the bond represented by — is present.

### EMBODIMENT 56

The method of any one of embodiments 36-54, wherein the bond represented by — is absent.

### EMBODIMENT 57

A compound obtained from the method of any one of embodiments 1-56.

### EMBODIMENT 58

A pharmaceutical composition comprising a pharmaceutically acceptable carrier and the compound of embodiment 57.

### EMBODIMENT 59

A cosmetic composition comprising a cosmetically acceptable carrier and the compound of embodiment 57.

### EMBODIMENT 60

A compound of embodiment 57 for use in medical therapy.

### EMBODIMENT 61

The use of a compound of embodiment 57 for the manufacture of a medicament for treating HIV, herpes, hepatitis, cancer, a viral infection, a fungal infection, a bacterial infection, or any combination thereof.

### EMBODIMENT 62

A method of treating a human afflicted with HIV, herpes, hepatitis, cancer, a viral infection, a fungal infection, a bacterial infection, or any combination thereof; the method comprising administering to a human in need of such treatment, an effective amount of the compound of embodiment 57.

### EMBODIMENT 63

A method of treating a human afflicted with HIV, the method comprising administering to a human in need of such treatment, an effective amount of the compound of embodiment 57.

### EMBODIMENT 64

A method of treating a human afflicted with herpes, the method comprising administering to a human in need of such treatment, an effective amount of the compound of embodiment 57.

### EMBODIMENT 65

A method of treating a human afflicted with hepatitis, the method comprising administering to a human in need of such treatment, an effective amount of the compound of embodiment 57.

### EMBODIMENT 66

A method of treating a human afflicted with cancer, the method comprising administering to a human in need of such treatment, an effective amount of the compound of embodiment 57.

### EMBODIMENT 67

A method of treating a human afflicted with a viral infection, the method comprising administering to a human in need of such treatment, an effective amount of the compound of embodiment 57.

### EMBODIMENT 68

A method of treating a human afflicted with a fungal infection, the method comprising administering to a human in need of such treatment, an effective amount of the compound of embodiment 57.

### EMBODIMENT 69

A method of treating a human afflicted with a bacterial infection, the method comprising administering to a human in need of such treatment, an effective amount of the compound of embodiment 57.

### EMBODIMENT 70

A method of treating a plant afflicted with a fungal infection, the method comprising administering to a plant in need of such treatment, an effective amount of the compound of embodiment 57.

### EMBODIMENT 71

A method of treating a plant afflicted with a bacterial infection, the method comprising administering to a plant in need of such treatment, an effective amount of the compound of embodiment 57.

### EMBODIMENT 72

A method of treating a plant afflicted with an insect infestation, the method comprising administering to a plant in need of such treatment, an effective amount of the compound of embodiment 57.

The invention can be illustrated by the following examples that do not limit in any manner the scope of the invention, as defined by the claims below.

### EXAMPLES

### Example 1

### 3-O-(3′,3′-Dimethylsuccinyl)betulinic aldehyde from betulinic aldehyde and 2,2-Dimethylsuccinic anhydride

2,2-Dimethylsuccinic anhydride (1 g, 4×2 mmol) was added to a stirred mixture of betulinic aldehyde (1 g, 2 mmol) and 4-dimethylaminopyridine (0.55 g, 2×2 mmol) in anhydrous pyridine (10 mL) at room temperature. The reaction mixture was stirred for 20 hours at 32° C. and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave white solids (0.88 g, 69% total yield).

¹H NMR (CDCl₃): 0.45-1.85 (complex CH—, CH₂, 23H) 0.79, 0.82, 0.84, 0.9, 0.97 (each 3H, s; 4-(CH₃)₂, 8-CH₃, 10-CH₃, 14-CH₃), 1.55 (6H, s, 3′-CH₃×2), 1.70 (3H, s, 20-CH₃), 2.03 (2H, m), 2.66, 2.59 (each 1H, d, H-2′), 2.86 (1H, m, H-19), 4.47 (1H, dd, H-3), 4.62, 4.75 (each 1H, br s, H-30), 9.44 (1H, s).

### Example 2

### 3-O-(3′,3′-dimethylsuccinyl)betulinic acid

Sodium chlorite (1 g, 9 mmol) and potassium phosphate monobasic (1.22 g, 9 mmol) in water (35 mL) was added dropwise to a stirred mixture of 3-O-(3′,3′-dimethylsuccinyl)betulinic aldehyde (0.88 g, 1.5 mmol), 2-methyl-2-butene (15 mL) and tert-butanol (50 mL). The mixture was stirred for 16 hours at room temperature, diluted with water (100 mL) and diethyl ether (50 mL). The organic layer was separated, dried with sodium sulfate and evaporated in vacuo to give crude product. This was recrystallized twice from hexane to give the product acid.

¹H NMR (pyridine-d5): 0.65-1.95 (complex CH-, CH₂, 22H) 0.73, 0.92, 0.97, 1.01, 1.05 (each 3H, s; 4-(CH₃)₂, 8-CH₃, 10-CH₃, 14-CH₃), 1.55 (6H, s, 3′-CH₃ .times.2), 1.80 (3H, s, 20-CH.sub.3), 2.24 (2H, m), 2.67 (2H, m), 2.89, 2.94 (each 1H, d, J=15.5 Hz, H-2′), 3.53 (1H, m, H-19), 4.76 (1H, dd, J=5.0, 11.5 Hz, H-3), 4.78, 4.95 (each 1H, br s, H-30). Total yield 0.65g (72%)

### Example 3

### 3-O-(3′,3′-dimethylsuccinyl)betulinic aldehyde from betulinic aldehyde and 2,2-dimethylsuccinic acid chloride

2,2-Dimethylsuccinic acid chloride (1.55 g, 4×2 mmol) was added to a stirred mixture of betulinic aldehyde (1 g, 2 mmol) and 4-(dimethylamino)pyridine (0.55 g, 2×2 mmol) in anhydrous pyridine (10 mL) at room temperature. The reaction mixture was stirred for 20 hours at 32° C. and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave white solids (0.8 g, 65% total yield).

¹H NMR (CDCl₃): 0.45-1.85 (complex CH-, CH₂, 23H) 0.79, 0.82, 0.84, 0.9, 0.97 (each 3H, s; 4-(CH₃)₂, 8-CH₃, 10-CH₃, 14-CH₃), 1.55 (6H, s, 3′-CH₃×2), 1.70 (3H, s, 20-CH₃), 2.03 (2H, m), 2.66, 2.59 (each 1H, d, H-2′), 2.86 (1H, m, H-19), 4.47 (1H, dd, H-3), 4.62, 4.75 (each 1H, br s, H-30), 9.44 (1H, s).

### Example 4

### 3-O-(3′,3′-dimethylsuccinyl)betulinic aldehyde from betulinic aldehyde and 2,2-dimethylsuccinic acid

2,2-Dimethylsuccinic acid (4 g, 15×2 mmol) was added to a stirred mixture of betulinic aldehyde (1 g, 2 mmol) and 4-(dimethylamino)pyridine (1.1 g, 4×2 mmol) in anhydrous pyridine (10 mL) at room temperature. The reaction mixture was reflux for 30 hours and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave white solids (0.85 g, 66% total yield).

¹H NMR (CDCl₃): 0.45-1.85 (complex CH—, CH₂, 23H) 0.79, 0.82, 0.84, 0.9, 0.97 (each 3H, s; 4-(CH₃)₂, 8-CH₃, 10-CH₃, 14-CH₃), 1.55 (6H, s, 3′-CH₃×2), 1.70 (3H, s, 20-CH₃), 2.03 (2H, m), 2.66, 2.59 (each 1H, d, H-2′), 2.86 (1H, m, H-19), 4.47 (1H, dd, H-3), 4.62, 4.75 (each 1H, br s, H-30), 9.44 (1H, s).

### Example 5

### 3-O-(3′,3′-dimethylsuccinyl)betulinic aldehyde from betulinic aldehyde and 2,2-dimethylsuccinic acid with DCC

To a solution of 2,2-dimethylsuccinic acid (0.62 g, 4.8 mmol) in DMF was added DCC (0.82 g, 4 mmol) at 0° C., and the mixture was stirred at room temperature for 5 hours. After N,N′-dicyclohexylurea was removed by filtration, betulinic aldehyde (1 g, 2 mmol) and 4-(dimethylamino)pylidine (0.55 g, 4 mmol) in anhydrous pyridine (10 mL) were added at 0° C., and the solution was stirred at 32° C. for 24 hours and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave white solids (0.93 g, 73% total yield).

¹H NMR (CDCl₃): 0.45-1.85 (complex CH—, CH₂, 23H) 0.79, 0.82, 0.84, 0.9, 0.97 (each 3H, s; 4-(CH₃)₂, 8-CH₃, 10-CH₃, 14-CH₃), 1.55 (6H, s, 3′-CH₃×2), 1.70 (3H, s, 20-CH₃), 2.03 (2H, m), 2.66, 2.59 (each 1H, d, H-2′), 2.86 (1H, m, H-19), 4.47 (1H, dd, H-3), 4.62, 4.75 (each 1H, br s, H-30), 9.44 (1H, s).

### Example 6

### 3-O-(3′,3′-dimethylsuccinyl)betulinic aldehyde from betulinic aldehyde and 2,2-dimethylsuccinic acid with acetic anhydride

A mixture of 2,2-dimethylsuccinic acid (0.62 g, 4.8 mmol) and acetic anhydride (2.5 g, 2 mmol) was heated at 100° C. for 1 hour. The acetic acid and acetic anhydride removed in vacuo, and the residue was added to a stirred mixture of betulinic aldehyde (1 g, 2 mmol) and 4-(dimethylamino)pyridine (0.55 g, 4 mmol) in anhydrous pyridine (10 mL) at room temperature. The reaction mixture was stirred for 20 hours at 32° C. and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave white solids (0.85 g, 67% total yield). ¹H NMR (CDCl₃): 0.45-1.85 (complex CH—, CH₂, 23H) 0.79, 0.82, 0.84, 0.9, 0.97 (each 3H, s; 4-(CH₃)₂, 8-CH₃, 10-CH₃, 14-CH₃), 1.55 (6H, s, 3′-CH₃×2), 1.70 (3H, s, 20-CH₃), 2.03 (2H, m), 2.66, 2.59 (each 1H, d, H-2′), 2.86 (1H, m, H-19), 4.47 (1H, dd, H-3), 4.62, 4.75 (each 1H, br s, H-30), 9.44 (1H, s).

### Example 7

### 3-O-(3′,3′-dimethylsuccinyl)betulinic acid from betulinic acid and 2,2-dimethylsuccinic acid chloride

2,2-Dimethylsuccinic acid chloride (1.55 g, 4×2 mmol) was added to a stirred mixture of betulinic acid (1 g, 2 mmol) and 4-(dimethylamino)pyridine (0.55 g, 2×2 mmol) in anhydrous pyridine (10 mL) at room temperature. The reaction mixture was stirred for 20 hours at 60° C. and cooled down to room temperature. The mixture was diluted with 5% HCl solution (20 mL) and dichloromethane (50 mL). The organic layer was separated, washed with 5% HCl solution (2×10 mL), water (2×20 mL), dried with sodium sulfate and concentrated under reduced pressure to give crude product. Crystallization from methanol gave colorless needles (0.91 g, 71% total yield).

¹H NMR (pyridine-d5): 0.65-1.95 (complex CH—, CH₂, 22H) 0.73, 0.92, 5 0.97, 1.01, 1.05 (each 3H, s; 4-(CH₃)₂, 8-CH₃, 10-CH₃, 14-CH₃), 1.55 (6H, s, 3′-CH₃×2), 1.80 (3H, s, 20-CH₃), 2.24 (2H, m), 2.67 (2H, m), 2.89, 2.94 (each 1H, d, J=15.5 Hz, H-2′), 3.53 (1 H, m, H-19), 4.76 (1H, dd, J=5.0, 11.5 Hz, H-3), 4.78, 4.95 (each 1H, br s, H-30).

### Example 8

### (3β)-lupan-3-ol-28-al from betulinic aldehyde

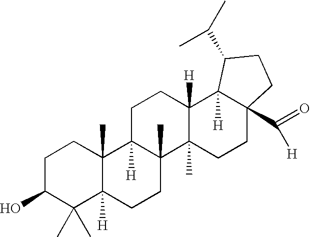

A solution of betulinic aldehyde (1 g, 2.28 mmol) in a mixture of THF and methanol (1:1, 10 mL) was hydrogenated under an H₂ atmosphere over 20% Pd/C (0.3 g, 20% wt) for 2 hours at room temperature and then filtered. After removal of the solvent in vacuum the crude product (dihydrobetulinic aldehyde) was obtained with 95% yield and a purity of about 93%.

¹H NMR (CDCl₃, 300 MHz) δ 9.63 (s, 1H), 3.2 (dd, J₁=10.1 Hz, J₂=5.5 Hz, 1H), 2.25-1.8 (m, 4H), 1.75-0.6 (m, 44H).

### Example 9

### 3β-3-(3′,3′′-dimethylsuccinyloxy)-lupan-28-al from dihydrobetulinic aldehyde

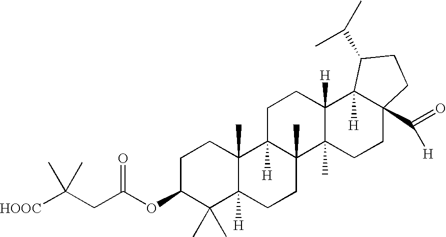

2,2-Dimethylsuccinic anhydride (2 g, 8×2 mmol) was added to a stirred mixture of dihydrobetulinic aldehyde (1 g, 2 mmol) and 4-dimethylaminopyridine (2.2 g, 8×2 mmol) in anhydrous pyridine 15 mL at room temperature. The reaction mixture was stirred for 48 hours at 60° C. and cooled down to room temperature. The mixture was diluted with 5% HCl solution (50 mL), the off-white precipitate was filtered off, washed with water (2×20 mL) and dried. Washing with hot methanol gave white solids (0.83 g, 67% total yield).

¹H NMR (CDCl₃): δ 9.63 (s, 1H), 4.5 (dd, J₁=10.1 Hz, J₂=5.5 Hz, 1H), 2.6 (m, 2H), 2.25-0.6 (m, 53H).

### Example 10

### 3β-3-glutaryloxy-lupan-28-al from dihydrobetulinic aldehyde

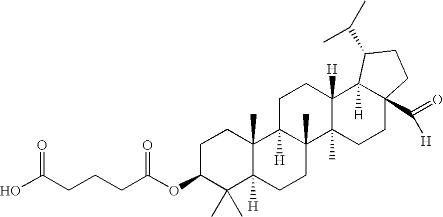

Glutaric anhydride (1 g, 4×2 mmol) was added to a stirred mixture of dihydrobetulinic aldehyde (1 g, 2 mmol) and 4-dimethylaminopyridine (0.55 g, 2×2 mmol) in anhydrous pyridine (10 mL) at room temperature. The reaction mixture was stirred for 24 hours at room temperature. The mixture was diluted with 5% HCl solution (20 mL), the precipitate was filtered off, washed with water (2×20 mL) and dried. Washing with hot methanol gave white solids (0.9 g, 69% total yield).

¹H NMR (CDCl³): δ 9.63 (s, 1H), 4.5 (dd, J₁=10.1 Hz, J₂=5.5 Hz, 1H), 2.5-2.3 (m, 4H), 2.28-0.7 (m, 49H).

### Example 11

### 3β-3-(3′-methylglutaryloxy)-lupan-28-al from dihydrobetulinic aldehyde

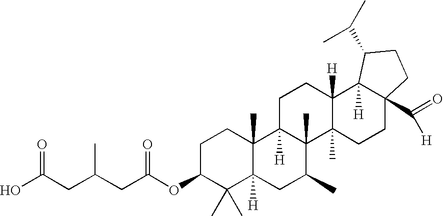

3-Methylglutaric anhydride (1 g, 4×2 mmol) was added to a stirred mixture of dihydrobetulinic aldehyde (1 g, 2 mmol) and 4-dimethylaminopyridine (0.55 g, 2×2 mmol) in anhydrous pyridine (10 mL) at room temperature. The reaction mixture was stirred for 24 hours at room temperature. The mixture was diluted with 5% HCl solution (20 mL), precipitate was filtered off, washed with water (2×20 mL) and dried. Washing with hot methanol gave white solids (1.07 g, 79% total yield).

¹H NMR (CDCl₃): δ 9.63 (s, 1H), 4.5 (dd, J₁=10.1 Hz, J₂=5.5 Hz, 1H), 2.5-2.3 (m, 2H), 2.25-0.8 (m, 53H).

### Example 12

### 3β-3-(3′,3′-tetramethylglutaryloxy)-lupan-28-al from dihydrobetulinic aldehyde

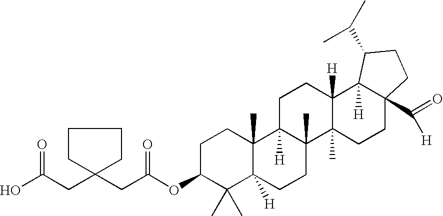

3,3-Tetramethyleneglutaric anhydride (1 g, 4×2 mmol) was added to a stirred mixture of dihydrobetulinic aldehyde (1 g, 2 mmol) and 4-dimethylaminopyridine (0.55 g, 2×2 mmol) in anhydrous pyridine (10 mL) at room temperature. The reaction mixture was stirred for 48 hours at room temperature. The mixture was diluted with 5% HCl solution (20 mL), the precipitate was filtered off, washed with water (2×20 mL) and dried. Washing with hot methanol gave white solids (0.96 g, 70% total yield).

¹H NMR (CDCl₃): δ 9.63 (s, 1H), 4.5 (dd, J₁=10.1 Hz, J₂=5.5 Hz, 1H), 2.6-2.45 (m, 4H), 2.35-0.7 (m, 55H).

### Example 13

### 3β-3-(3′,3′-pentamethylglutaryloxy)-lupan-28-al from dihydrobetulinic aldehyde

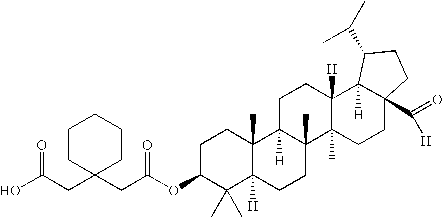

1,1-Cyclohexanediacetic acid chloride (2 g, 8×2 mmol) was added to a stirred mixture of dihydrobetulinic aldehyde (1 g, 2 mmol) and 4-dimethylaminopyridine (2.2 g, 8×2 mmol) in anhydrous pyridine (15 mL) at room temperature. The reaction mixture was stirred for 48 hours at 65° C. and cooled down to room temperature. The mixture was diluted with 5% HCl solution (50 mL), the off-white precipitate was filtered off, washed with water (2×20 mL) and dried. Washing with hot methanol gave white solids (0.83 g, 67% total yield).

¹H NMR (CDCl₃): δ 9.63 (s, 1H), 4.5 (dd, J₁=10.1 Hz, J₂=5.5 Hz, 1H), 2.55 (m, 4H), 2.35-0.7 (m, 57H).

### Example 14

### 3β-3-(mono-Ethylsuccinyloxy)-lupan-28-al from dihydrobetulinic aldehyde

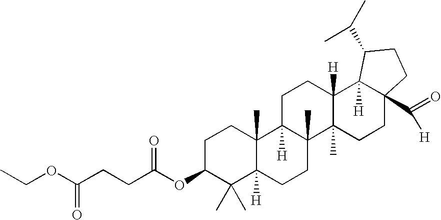

mono-Ethylsuccinate chloride (1 g, 4×2 mmol) was added to a stirred mixture of dihydrobetulinic aldehyde (1 g, 2 mmol) and 4-dimethylaminopyridine (0.55 g, 2×2 mmol) in anhydrous pyridine (10 mL) at room temperature. The reaction mixture was stirred for 48 hours at room temperature. The mixture was diluted with CH₂Cl₂ (80 mL). The CH₂Cl₂ solution was washed with 5% HCl solution (2×30 mL), and H₂O(2×25 mL), and dried over Na₂SO₄. The dark brown residue after solvent evaporation was purified by washing with hot methanol (2×20 mL), and gave off-white solids (1.09 g, 81% total yield).

¹H NMR (CDCl₃): δ 9.63 (s, 1H), 4.5 (dd, J₁=10.1 Hz, J₂=5.5 Hz, 1H), 4.15 (m, 2H), 2.96 (m, 1H), 2.65 (s, 3H), 2.35-0.7 (m, 49H).

### Example 15

### 3β-3-( mono-Ethylglutaryloxy)-lupan-28-al from dihydrobetulinic aldehyde

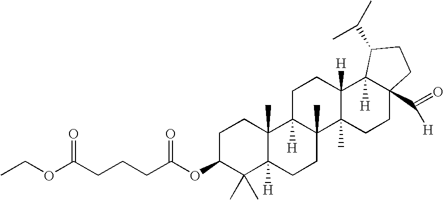

Ethyl hydrogen glutarate chloride (1 g, 4×2 mmol) was added to a stirred mixture of dihydrobetulinic aldehyde (1 g, 2 mmol) and 4-dimethylaminopyridine (0.55 g, 2×2 mmol) in anhydrous pyridine (10 mL) at room temperature. The reaction mixture was stirred for 48 hours at room temperature. The mixture was diluted with CH₂Cl₂ (80 mL). The CH₂Cl₂ solution was washed with 5% HCl solution (2×30 mL), and H₂O (2×25 mL), and dried over Na₂SO₄. The dark brown residue after solvent evaporation was purified by washing with hot methanol (2×20 mL), and gave off-white solids (1.18 g, 86% total yield).

¹H NMR (CDCl₃): δ 9.63 (s, 1H), 4.5 (dd, J₁=10.1 Hz, J₂=5.5 Hz, 1H), 4.15 (m, 2H), 2.85-0.7 (m, 55H).

### Example 16

### Dihydrobetulinic acid from betulinic acid

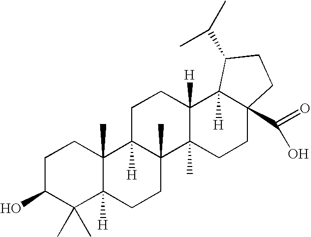

A solution of betulinic acid (1 g, 2.2 mmol) in a methanol (50 mL) was hydrogenated under a H₂ atmosphere over 20% Pd/C (0.3 g, 20% wt) for 5 hours at room temperature and then filtered. After removal of the solvent in vacuum the crude product was crystallized from MeOH. After filtration white crystals were obtained with 90% yield and purity of about 94%.

¹H NMR (CDCl₃, 300 MHz) δ 3.2 (dd, J₁=10.1 Hz, J₂=5.5 Hz, 1H), 2.2 (m, 3H), 1.95-0.6 (m, 44H).

The use of a methanol/THF solvent system provided similar results to the use of a methanol-only solvent system. In methanol, the solubility of betulinic acid is lower than in a MeOH/THF mixture. Accordingly, the reaction time is dependant upon the amount of methanol in the reaction mixture.

All literature and patent citations above are hereby expressly incorporated by reference at the locations of their citation. Specifically cited sections or pages of the above cited works are incorporated by reference with specificity. The invention has been described in detail sufficient to allow one of ordinary skill in the art to make and use the subject matter of the following Embodiments. It is apparent that certain modifications of the methods and compositions of the following Embodiments can be made within the scope and spirit of the invention.

1. A method for preparing a compound of formula (I): 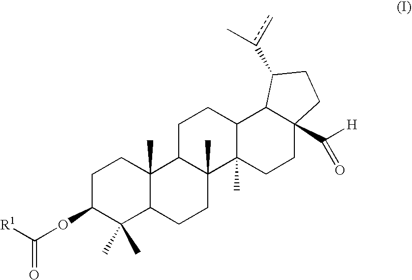
the method comprising contacting a compound of formula (II): 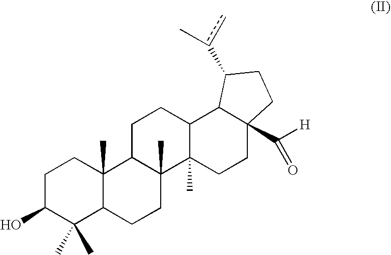
with an effective amount of a compound of formula (III) or (IV): 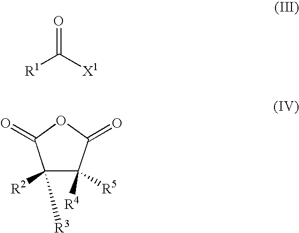
wherein,
R¹ is X¹C(═O)R^{x}—;
R^{x} is alkylene, cycloalkylene, carbocyclene, arylene, heterocyclene, or heteroarylene;
X¹ is hydroxyl, halo, alkoxy or —OC(═O)R^{y};
R^{y} is alkyl, cycloalkyl, carbocycle, aryl, heterocycle, or heteroaryl;
each of R²-R⁵ is independently H, alkyl, cycloalkyl, carbocycle, aryl, heterocycle, or heteroaryl; and
the bond represented by — is optionally present.

2. The method of claim 1, wherein
R¹ is HOOCC(CH₃)₂CH₂—, BrOCC(CH₃)CH₂—, or ClOCC(CH₃)₂CH₂—;
R^{x} is —C(CH₃)₂CH₂—;
X¹ is hydroxyl, bromo, chloro, or —OC(═O)R^{y}; and
each R², R³, R⁴, and R⁵ is independently methyl or hydrogen.

3-25. (canceled)

26. The method of claim 1, further comprising contacting the compound of formula (I) with an effective amount of NaClO₂, KClO₂, or a combination thereof, to provide a compound of formula (V): 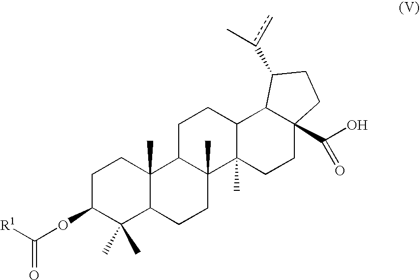
or a pharmaceutically acceptable salt thereof.

27-35. (canceled)

36. A method for preparing a compound of formula (VI): 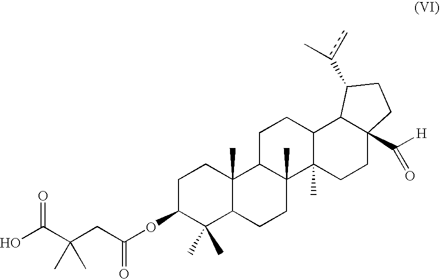
the method comprising contacting a compound of formula (II): 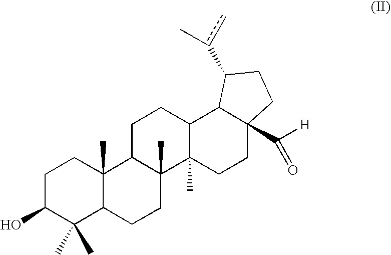
with an effective amount of a compound selected from the group of 2,2-dimethylsuccinic acid, 2,2-dimethylbutanedioyl dichloride, 2,2-dimethylbutanedioyl dibromide, and 2,2-dimethylsuccinic anhydride;
wherein the bond represented by — is optionally present.

37. (canceled)

38. The method of claim 36, wherein the contacting is carried out in a solvent system selected from the group of ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

39-42. (canceled)

43. The method of claim 36, further comprising contacting the compound of formula (VI) with an effective amount of NaClO₂ or KClO₂, to provide a compound of formula (VII): 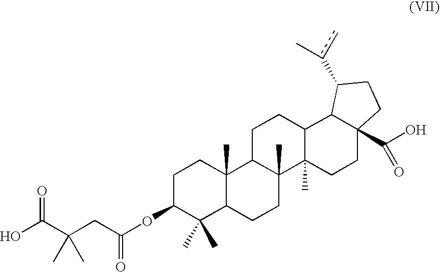
or a pharmaceutically acceptable salt thereof.

44. The method of claim 43, wherein the contacting is carried out at a temperature of about 10° C. to about 120° C.

45. The method of claim 43, wherein the contacting is carried out in a solvent system selected from the group of water, an alcohol, unsaturated hydrocarbons, ether, DMF, DMAA, DMSO, xylene, toluene, pyridine, chloroform, methylene chloride, dioxane, mineral oil, ethyl acetate, benzene, morpholine, pyrrole, cyclohexane, cyclohexanone, acetone, and pyrrolidinone.

46. The method of claim 43, further comprising a free halogen scavenger that is an unsaturated hydrocarbon selected from the group of amylene, cyclohexene, methylcyclohexene and cyclopentene.

47-48. (canceled)

49. The method of claim 43, wherein the contacting is carried out for a period of time of about 30 minutes to about 48 hours.

50. The method of claim 43, wherein at least about 10 kg of the compound of formula (VII) is obtained.

51. The method of claim 43, wherein at least about 85 mol % of the compound of formula (VII) is obtained, based upon the compound of formula (VI).

52. The method of claim 43, wherein the compound of formula (VII) is obtained having a purity of at least about 95 wt. %.

53. The method of claim 43, wherein the compound of formula (VI) is contacted with an effective amount of NaClO₂ or KClO₂, in the presence of a basic catalyst selected from the group of amines, alkylamines, dialkylamines, trialkylamines, pyridine, N,N-dimethylaminopyridine, triethylamine, 2,4,6-collidine, 2,6-lutidine, morpholine, imidazole, PPY(4-pyrrolidinopyridine), and DABCO (1,4-diazabicyclo(2,2,2)octane).

54. The method of claim 43, wherein the compound of formula (VI) is contacted with an effective amount of NaClO₂ or KClO₂, in the presence of a condensation catalyst selected from the group of DCC (N,N-dicyclohexylcarbodiimide), 2,4,6-trichlorobenzoyl chloride, di-2-pyridyl carbonate, diethyl azodicarboxylate and triethylphosphite, 1,2-benzisoxazol-3-yl-diphenylphosphate, N,N-carbonyldiimidazole and 1,8-diazabicyclo[5,4,0]-undec-7-ene, isoureas, benzoxazoles, and benzisothiazoles.

55. The method of claim 36, wherein the bond represented by — is present.

56. The method of claim 36, wherein the bond represented by — is absent.

57. A compound obtained from the method of claim 1.

58. A pharmaceutical composition comprising a pharmaceutically acceptable carrier and the compound of claim 57.

59. A cosmetic composition comprising a cosmetically acceptable carrier and the compound of claim 57.
